(19) United States
(12) Patent Application Publication
(10) Pub. No.: US 2005/0232947 A1
(43) Pub. Date: Oct. 20, 2005

(54) Bacterial spores

(76) Inventor: Simon Michael Cutting, Surrey (GB)

Correspondence Address: THE MAXHAM FIRM, 750 "B" STREET, SUITE 3100, SAN DIEGO, CA, 92101, US

(21) Appl. No.: 10/506,869

(22) PCT Filed: Mar. 7, 2003
(86) PCT No.: PCT/GB03/00989

§ 371 Date: Apr. 11, 2005

(30) Foreign Application Priority Data
Mar. 7, 2002 (GB) 0205378.3

(51) Int. Cl. A61K 39/02; C12N 15/74; C12N 1/21

(52) U.S. Cl. 424/200.1; 435/252.3; 435/471

(57) ABSTRACT

The invention provides a spore genetically modified with genetic code comprising at least one genetic construct encoding an antigen and a spore coat protein as a chimeric gene, said genetically modified spore having said antigen expressed as a fusion protein with said spore coat protein.

### BACKGROUND OF THE INVENTION

1. Field of the Invention

This invention relates to the use of spores in eliciting an immune response, a method of eliciting said immune response and to a method of making said spores.

2. Discussion of Related Art

Infection is the leading cause of death in human populations. The two most important contributions to public health in the past 100 years have been sanitation and vaccination, which together have dramatically reduced deaths from infectious disease.

The development of improved vaccination strategies has always been of the utmost importance for a number of reasons.

Firstly, to provide better levels of immunity against pathogens which enter the body primarily through the mucosal surfaces. Vaccines are generally given parentally. However, many diseases use the gastrointestinal (GI) tract as the primary portal of entry. Thus, cholera and typhoid are caused by ingestion of the pathogens Salmonella typhi and Vibrio cholera and subsequent colonisation at (V. cholera) or translocation (S. typhi) across the mucosal epithelium (lining the GI tract). Similarly, TB is initially caused by infection of the lungs by Mycobacterium tuberculi. Immunisation via an injection generates a serum response (humoral immunity) which includes a predominant IgG response which is least effective in preventing infection. This is one reason why many vaccines are partially effective or give short protection times.

Secondly, to provide needle-less routes of administration. A major problem of current vaccination programmes is that they require at least one injection (for example tetanus vaccine). Although protection lasts for 10 years, children are initially given three doses by injection and this should be followed by a booster every 5 years. In developed countries many people will choose not to take boosters because of ‘fear of injection’. In contrast, in developing countries where mortality from tetanus is high the problems lie with using needles that are re-used or are not sterile.

Thirdly, to offer improved safety and the minimisation of adverse side effects. Many vaccines consist of live organisms which are either rendered non-pathogenic (attenuated) or are inactivated in some way. While in principle, this is considered safe there is evidence showing that safer methods must be developed. For example, in 1949 (the Kyoto incident) 68 children died from receiving a contaminated diphtheria vaccine (Health 1996). Likewise, in the Cutter incident of 1995, 105 children developed polio. It was found that the polio vaccine had not been correctly inactivated with formalin. Many other vaccines, for example the MMR (measles-mumps-rubella) vaccine and the whooping cough vaccine (Health, 1996) are plagued with rumours of side effects.

Fourthly, to provide economic vaccines for developing countries where poor storage and transportation facilities prevent effective immunisation programmes. In developing countries where a vaccine must be imported it is assumed that the vaccine will be stored and distributed correctly. The associated costs of maintaining vaccines in proper hygienic conditions under refrigeration are significant for a developing country. For some vaccines such as the oral polio vaccine and BCG vaccine the vaccines will only survive for one year at 2-8° C. (Health, 1996). The need for a robust vaccine that can be stored indefinitely at ambient temperature is a high priority now for developing countries. This type of vaccine should ideally be heat stable, able to withstand great variations in temperature as well as desiccation. Finally, a vaccine that is simple to produce would offer enormous advantages to a developing country and would potentially be producable in that country.

### SUMMARY OF THE INVENTION

A way of ameliorating these problems has been sought.

Accordingly, the present invention provides a spore genetically modified with genetic code comprising at least one genetic construct encoding an antigen and a spore coat protein as a chimeric gene, said genetically modified spore having said antigen expressed as a fusion protein with said spore coat protein.

It is an advantage of the present invention in that the use of spores to administer vaccines will eliminate the need for injections and the problems associated with needles in developing countries. In addition to this, spores are stable and are resistant to heat and desiccation, therefore overcoming problems of storing vaccines in developing countries. Spores are easy to produce, and can be done at low cost making the production of vaccines in accordance with the invention economical and finally, as a non-pathogen and its current use as an oral probiotic, the use of Bacillus subtilis makes this a safer vaccine system than those currently available.

It is a further advantage of the invention that the spores elicit an immune response at the mucosal membranes. This makes the vaccination more effective against mucosal pathogens e.g. S. typhi, V. cholera and M. tuberculi.

A vaccine delivered at the mucosal surfaces will be more effective in combating those diseases which infect via the mucosal route. The mucosal routes of vaccine administration would include oral, intra-nasal and/or rectal routes.

Preferably the spore is of Bacillus species.

Preferably the vegetative cell is of Bacillus species.

The genetic code comprises DNA or cDNA. It will be appreciated that the term ‘genetic-code’ is intended to embrace the degeneracy of codon usage.

The genetic construct preferably comprises at least part of a spore coat protein gene and at least part of an antigen gene, in the form of a chimeric gene.

The antigen gene is preferably located at the 3′ end of the spore coat protein gene. Alternatively the antigen gene may be located at the 5′ end of the spore coat protein gene or internally of the spore coat protein gene.

Preferably the genetic construct comprises a spore coat promoter at the 5′ end of the chimeric gene.

The genetic construct comprises a plasmid or other vector wherein the chimeric gene is located in a multiple cloning site flanked by at least part of an amyE gene. Alternatively, the genetic construct comprises a plasmid or other vector wherein the chimeric gene is located in a multiple cloning site flanked by at least part of a thrC gene. It will be appreciated that the invention is not limited to insertion at amyE and thrC genes. Insertion into any gene is permissible as long as the growth and sporulation of the organism is not impaired i.e. the insertion is functionally redundant

Preferably the genetic construct is used to transform a vegetative mother cell by double crossover recombination. Alternatively the genetic construct is an integrative vector e.g. p JH101 which is used to transform the vegetative mother cell by single crossover recombination.

The antigen is preferably at least one of tetanus toxin fragment C or labile toxin B subunit. Alternatively the antigen may be any antigen, adapted, in use, to elicit an immune response.

The spore coat protein is preferably cotB. Alternatively the spore coat protein is selected from the group consisting of cotA, cotC, cotD, cotE and cotF. Alternatively the spore coat protein is selected from the group consisting of cotG, cotH, cotJA, cotJC, cotM, cotSA, cotS, cotT, cotV, cotW, cotX, cotY and cotZ.

The spores may be administered by an oral or intranasal or rectal route. The spores may be administered using one or more of the said oral or intranasal or rectal routes.

Oral administration of spores may be suitably via a tablet a capsule or a liquid suspension or emulsion. Alternatively the spores may be administered in the form of a fine powder or aerosol via a Dischaler® or Turbonhaler®.

Intranasal administration may suitably be in the form of a fine powder or aerosol nasal spray or modified Dischaler® or Turbohaler®.

Rectal administration may suitably be via a suppository.

The spores according to the invention are heat inactivated prior to administration such that they do not germinate into vegetative cells.

According to a further aspect the present invention provides a genetically modified spore according to the invention for use as an active pharmaceutical substance.

According to a further aspect the present invention provides at least two different genetically modified spores, the or each modified spore expressing at least one different antigen, according to the invention for use as active pharmaceutical compositions.

According to a further aspect, the present invention provides a method of producing a genetically modified spore, which method comprises the steps;
producing genetic code comprising at least one genetic construct encoding an antigen and a spore coat protein as a chimeric gene;
using said at least one genetic construct to transform a vegetative mother cell;
inducing said transformed mother cell to sporulate;
isolating the resulting genetically modified spores.

The spores are heat inactivated prior to administration such that they do not germinate into vegetative cells.

According to a further aspect, the present invention provides a composition comprising a genetically modified spore, according to the invention, in association with a pharmaceutically acceptable excipient or carrier.

Suitable pharmaceutically acceptable carriers would be well known to a person of skill in the art and would depend on whether the pharmaceutical composition was intended for oral, rectal or nasal administration.

According to a further aspect the present invention provides a genetically modified spore according to the invention for use in a method of medical treatment.

According to a further aspect, the present invention provides a genetically modified spore according to the invention for use in the manufacture of a medicament, for use in a method of medical treatment.

A method of medical treatment is preferably immunising a human or animal against a disease by administering a vaccine.

According to a further aspect, the present invention provides a method of medical treatment, which method comprises the steps of;
orally or intra-nasally or rectally administering a genetically modified spore according to invention to a human or animal in need of medical treatment;
said genetically modified spore eliciting an immune response for use in the prevention of a disease.

### BRIEF DESCRIPTION OF THE DRAWING

The invention will now be described merely by way of example, with reference to the accompanying figures, of which:

FIG. 1 shows detection of presence of CotB and TTFC by immunofluorescence. Sporulation of B. subtilis strains was induced by the resuspension method (1), and samples were taken 5 h after the onset of sporulation. Samples were labelled with rabbit anti-CotB and mouse anti-TTFC antisera, followed by anti-rabbit IgG-FITC (green fluorescein, Panels A & C) and anti-mouse IgG-TRITC (red fluorescein, Panels B & D) conjugates. Panels A & B, PY79 (wild type); Panels C & D, RH103 (CotB-TTFC expressing strain).

FIG. 2 shows systemic responses after mucosal immunisations. Serum anti-TTFC specific IgG responses following oral (Panel A) or intranasal (Panel B) immunisations with recombinant B. subtilis spores expressing CotB-TTFC. Groups of seven mice were immunised (↑) orally or intranasally with spores expressing CotB-TTFC (RH103; ∘) or non-recombinant spores (PY79; ∘). A dose of 1.67×10¹⁰ spores was used for each oral dose and 1.1×10⁹ for the intranasal route and individual serum samples from groups were tested by ELISA for TTFC-specific IgG. Sera from a naive control group (Δ) and a group orally immunised with 4 mg/dose of purified TTFC protein (⋄) were also assayed. Data are presented as arithmetic means and error bars are standard deviations.

FIG. 3 shows antibody isotype profiles. Anti-TTFC antibody isotype profiles on day 54 post oral immunisation or day 48 post intranasal immunisation with recombinant spores expressing CotB-TTFC (RH103) or non-recombinant (PY79) B. subtilis spores as described in the legends to FIG. 2A and FIG. 2B. TTFC-specific IgG1, IG2a, IgG2b, IgG3, IgM and IgA isotypes were determined by indirect ELISA. Sera from a naïve control group were also assayed. The end-point titer was calculated as the dilution of serum producing the same optical density as a 1/40 dilution of a pooled pre-immune serum. Data are presented as arithmetic means and error bars are standard deviations.

FIG. 4 shows TTFC-specific fecal IgA responses. Groups of seven mice were immunized orally (Panel A) or intranasally (Panel B) with recombinant spores expressing CotBTTFC (RH103) or non-recombinant spores (PY79) as described in the legends to FIGS. 2A and 2B respectively. Fresh fecal pellets were collected from these immunised mice as well as a naive group and tested for the presence of TTFC-specific IgA as described in the Materials and Methods section of Example 2. The end-point titer was calculated as the dilution of the fecal extract producing the same optical density as the undiluted pre-immune fecal extract. Data are presented as arithmetic means and error bars are standard deviations.

FIG. 5 shows Anti-spore serum IgG and mucosal IgA responses. Groups of seven mice were immunised (↑) by the oral (Panels A and B) or intranasal routes (Panels C and D) as described in the legend to FIG. 2 with recombinant spores expressing CotB-TTFC (∘) or non-recombinant spores (o). Individual samples were tested by indirect ELISA for B. subtilis spore coat-specific serum IgG (Panels A and C) or spore coat-specific fecal IgA (Panels B and D). Sera from a naïve group (Δ) was also assayed. The end point IgG titer was calculated as the dilution of serum producing the same optical density as a 1/40 dilution of a pooled pre-immune serum. The end-point IgA titer was calculated as the dilution of the fecal extract producing the same optical density as the undiluted preimmune fecal extract. Data are presented as arithmetic means and error bars are standard deviations.

### DETAILED DESCRIPTION OF THE PREFERRED EMBODIMENTS

The invention will now be illustrated with reference to the following non-limiting Examples.

### EXAMPLE 1

Chimeric genes were constructed in which TTFC or LTB gene sequences were fused, in frame, to a specific cot gene. The constructs were then introduced into the chromosome of B. subtilis. Expression of the chimeric genes was then confirmed and immunisations were performed using inbred mice (Black C57 inbreds). Immune responses were then measured. Unless otherwise stated, cot genes refers to cotA, cotB, cotC, cotD, cotE and cotF.

TABLE 1
Recombinant chimeric genes

| TTFC¹ | LTB² | TTFC & LTB |
|---|---|---|
| cotA-TTFC | CotA-LTB | cotA-LTB cotB-TTFC |
| cotB-TTFC | CotB-LTB | CotA-LTB cotE-TTFC |
| cotC-TTFC | CotC-LTB | cotA-LTB cotD-TTFC |
| cotD-TTFC | CotD-LTB | |
| cotE-TTFC | CotE-LTB | |
| cotF-TTFC | CotF-LTB | |

¹placed at the amyE locus
²placed at the thrC locus

a) Construction of Chimeric Genes

PCR (polymerase chain reaction) was used to amplify the specific cot gene to enable the 3′-end of the amplified cot gene sequence to be fused to the 5′-end of a similar PCR product carrying the 5′-end of TTFC or LTB. Ligation PCR products was achieved by restriction digest of the PCR products. This was enabled by PCR amplification using primers carrying embedded restriction sites. Appropriate cloning vectors (see below) were restricted (cut) with restriction enzymes recognising the 5′-end of the cot gene and the 3′-end of the antigen gene. The cleaved PCR products were ligated with cleaved vector and recombinants assessed using standard techniques known to those in the art.

(In this process it is essential that the cot gene carries its own promoter sequences at the 5′-end of the gene.)

b) Vectors for Chromosomal Insertion

The essential features of the vector pDG364 are the right and left flanking arms of the amyE gene (referred to as amyE front and amyE back). Cloned DNA (i.e. the cot-antigen chimera) is introduced into the multiple cloning sites using general PCR techniques, the clone is then validated and the selected plasmid clone linearised by digestion with enzymes recognising the relevant backbone sequences (e.g. PstI). The linearised DNA is now used to transform competent cells of B. subtilis. Transformants are selected by using an antibiotic resistance gene carried by the plasmid (chloramphenicol resistance). The linearised plasmid will only integrate via a double crossover recombination event using the front and back flanking arms of amyE for recombination. In the process the cloned DNA is introduced into the amyE gene and the amyE gene inactivated in the process. This procedure minimises damage to the chromosome and does not impair cell growth, metabolism or spore formation. Since the inserted gene chimera is at the amyE locus in the chromosome the gene is in trans to the normal cot genetic locus. For example, when the cotA gene is fused to TTFC and introduced into the amyE locus, there also exists a normal cotA gene elsewhere in the chromosome. Thus, the cell is now partially diploid, it carries one normal cotA gene and one chimeric gene.

In addition to pDG364, another suitable vector is pDG1664. This vector is almost identical to pDG364 but differs by the following;
i) it carries the erythromycin-resistance gene, erm. This enables selection of transformed B. subtilis cells using erythromycin instead of chloramphenicol, and
ii) instead of the front and back portions of the amyE gene it carries the front and back portions of the thrC gene. thrC is redundant.

A final route for cloning is to use an integrational vector. Many such vectors exist, but pSGMU2 or pJH101 are preferred. In this method, the cot gene in the clone and the resident chromosomal cot gene would introduce a cot-antigen chimera into the chromosome by virtue of homology shared. Following single crossover recombination the entire plasmid with the cot-antigen chimera is introduced into the chromosome at the chromosomal position of the cot gene. Thus, in doing so, the resident cot gene is modified. This is in contrast to the pDG364/pDG1664 vectors which are placed elsewhere and do not modify the resident cot gene.

c) Multiple Antigen Presentation

To achieve multiple antigen presentation on the spore coat it is necessary to use two different plasmid vectors, for example pDG364 and pDG1664. One chimeric gene is made in pDG364 and the chimera introduced at the amyE locus and a second chimera made in pDG1664 and introduced at the thrC locus. In this case each transformational event requires separate antibiotic resistance selection. It will be appreciated that any relevant technology known to those of skill in the art could be applied to create multiple antigen presentation on the spore coat.

d) Validation of Strains

Isogenic strains carrying the chimeras shown in Table 1 were validated for expression of a foreign antigen. Specifically, strains were grown and induced to sporulate using established procedures. Spores at about hour 20-24 following the induction of sporulation were harvested and total spore coat proteins recovered using ether SDS-DTT extraction or NaOH extraction. Western blotting using anti-TTFC or anti-LTB antibodies was used to demonstrate the presence of the foreign antigen. Levels of protein were generally lower in the cotE and cotF chimeras. The validation confirmed that these antigens were not subject to inadvertent proteolysis or degradation.

TTFC can be expressed at the thrC locus and LTB from the amyE locus with identical levels of gene expression.

Final validation of strains involved assessing whether the spore's resistance properties had been affected in any way. Spore suspensions of each strain were prepared (shown in Table 1). These spore suspensions were heated at 80° C. for 30 min and shown to carry approximately the same number of viable spore units before and after heat treatment. The expression of the foreign antigen had no effect on spore resistance properties.

e) Intra-Peritoneal Immunisation

Spores were prepared from each of the recombinant strains shown in table 1 and the suspensions were purified by repeated washing to remove contaminating vegetative cells. The suspensions were then heat-treated at 65° C. to inactivate any residual vegetative (unsporulated cells) and subsequently used to dose mice via an intra-peritoneal route at a dose of 1×10⁹ spores/ml on days 0, 14 and 28. Serum samples were taken thereafter and antibody titres determined by ELISA. All constructs gave high levels of serum IgG compared to naïve mice or mice immunised with non-recombinant spores. These results showed that both TTFC and LTB chimeras are immunogenic and are capable of eliciting an immune response.

f) Mucosal Immunity

To achieve mucosal immunity two approaches were used; oral dosing and intranasal dosing. For oral administration of spores expressing TTFC fusion proteins, 1×10¹⁰ spores/dose were administered by intra-gastric lavage to black C57 inbred mice using multiple doses over a 35 day period. Tail bleeds and fecal samples were taken at appropriate times and analysis made for serum IgG in tail bleeds and IgA in fecal samples. High levels of anti-TTFC IgG and IgA were found. Similar high levels of immunity (both IgG and IgA) following oral immunisation of mice with spores expressing LTB (not shown) have been observed.

Similarly, intranasal dosing of mice with spores expressing LTB was achieved using 1×10⁹ spores/dose using micropipettes to administer spores (20 μl) on days 0, 14 and 28. High levels of mucosal immunity were generated demonstrating the potential of spores as mucosal vaccine vehicles using the intra-nasal route for delivery. We have observed similar high levels of immunity (both IgG and IgA) following intranasal immunisation of mice with spores expressing TTFC.

Using spores expressing both TTFC and LTB we were able to achieve similarly high levels of anti-TTFC and anti-LTB IgG and IgA following oral and intranasal immunisation.

g) dosage

In pilot studies we know that about 1×10⁹ spores/dose is the minimum dose of spores required for oral immunisation and _{1×10}8 spores/dose for intranasals. It is possible that with alternative dosing regimes (of which there are many) a lower dose could be used.

Spores according to the invention could be used to display any biogically active molecule. For example, an enzyme for an industrial application.

Any spore forming species could be used for heterologous antigen presentation. However, other spore-forming micro-organisms are unlikely to carry the same complement of spore coat proteins. Indeed, some spore formers such as Bacillus cereus may contain only one cat protein. However, using antisera to cotA, cotB, cotC, cotD, cotE and cotF in our collection it would be possible to identify homologous or cross-reacting coat proteins from the coats of spore formers and then clone the genes by reverse genetics.

Spores according to the invention could also be used with adjuvants. These might include cholera toxin, chitosan or aprotonin.

### EXAMPLE 2

Materials and Methods:

Preparation of Spores

B. subtilis strain RH103 (amyE::cotB-tetC) was used for all immunisations together with its isogenic ancestor, PY79 (2). RH103 has been described elsewhere (3) and carries a fusion of tetanus toxin fragment C (TTFC; 47 kDa) to the C-terminus of the outer spore coat protein CotB (59 kDa). The chimeric cotB-tetC gene was carried at the amyE locus of B. subtilis and was therefore in trans to the endogenous cotB gene. Sporulation of either RH103 or PY79 was made in DSM (Difco-sporulation media) media using the exhaustion method as described elsewhere (1). Sporulating cultures were harvested 22 h after the initiation of sporulation. Purified suspensions of spores were made as described by Nicholson and Setlow (1) using lysozyme treatment to break any residual sporangial cells followed by washing in 1 M NaCl, 1 M KCl and water (two-times). PMSF was included to inhibit proteolysis. After the final suspension in water spores were treated at 68° C. for 1 h to kill any residual cells. Next, the spore suspension was titred immediately for CFU/ml before freezing at −20° C. Using this method we could reliably produce 6×10¹⁰ spores per litre of DSM culture. Each batch of spores prepared in this way was checked for the presence of the 106 kDa hybrid CotB-TTFC protein in extracts of spore coat protein by Western blotting using a polyclonal TTFC antiserum.

Immunofluorescence Microscopy

B. subtilis strains (PY79, RH103) were induced to sporulate by the resuspension method (1). Samples were collected at defined times after the onset of sporulation and fixed directly in the resuspension medium using the procedure described by Harry et al (4) with the following modifications. After suspension in GTE-lysozyme (50 mM glucose, 20 mM Tris-HCl pH 7.5, 10 mM EDTA, lysozyme 2 mg/ml), samples (10 μl) were immediately applied on microscope cover glasses (BDH) that had been treated with 0.01% (w/v) poly-L-lysine (Sigma). After 4 min, the liquid was aspirated from the cover glass, which was then allowed to dry completely for 2 h at room temperature. The glass was washed 3 times in PBS pH 7.4, blocked for 15 min with 2% BSA in PBS at room temperature, then washed 9 more times. Samples were incubated with rabbit anti-CotB and mouse anti-TTFC sera for 45 min at room temperature, washed 3 times, then incubated further with anti-rabbit IgG-FITC and anti-mouse IgG-TRITC conjugates (Sigma) for 45 min at room temperature. After 3 washings, the cover glass was mounted onto a microscope slide and viewed under a Nikon Eclipse E600 fluorescence microscope. Images were captured using a Nikon DMX1200 digital camera, processed with the Lucia GF software, and saved in TIFF format.

TTFC Protein

Recombinant TTFC was produced in E. coli BL21 (DE3 pLys) from a pET28b expression vector (Novagen) that carried the tetC gene fused to a C-terminal polyhistidine tag. High levels of expression were obtained upon induction of the bacteria, and purification of TTFC was by passage of a cell lysate through a nickel affinity column.

Eluted TTFC-His protein was checked for integrity by SDS-PAGE and the concentration determined using the BioRad DC Protein Assay kit.

Indirect ELISA for Detection of Antigen-specific Serum and Mucosal Antibodies

Plates were coated with 50 μl/well of the specific antigen (2 μg/ml in carbonate/bicarbonate buffer) and left at room temperature overnight. Antigen was either extracted spore coat protein or purified TTFC protein. After blocking with 0.5% BSA in PBS for 1 h at 37° C. serum samples were applied using a 2-fold dilution series starting with a 1/40 dilution in ELISA diluent buffer (0.1M Tris-HCl, pH 7.4; 3% (w/v) NaCl; 0.5% (w/v) BSA; 10% (v/v) sheep serum (Sigma); 0.1% (v/v) Triton-X-100; 0.05% (v/v) Tween-20). Every plate carried replicate wells of a negative control (a 1/40 diluted preimmune serum), a positive control (serum from mice immunised parentally with either TTFC protein or spores). Plates were incubated for 2 h at 37° C. before addition of antimouse HRP conjugates (all obtained from Sigma with the exception of Serotec for the subclasses). Plates were incubated for a further 1 h at 37° C. then developed using the substrate TMB (3, 3′, 5, 5′-tetramethyl-benzidine; Sigma). Reactions were stopped using 2 M H₂SO₄. Dilution curves were drawn for each sample and endpoint titres calculated as the dilution producing the same optical density as the 1/40 dilution of a pooled preimmune serum. Statistical comparisons between groups were made by the Mann-Whitney U test. A p value of >0.05 was considered non-significant. For ELISA analysis of fecal IgA, we followed the procedure of Robinson et al (5) using approximately 0.1 g fecal pellets that had been suspended in PBS with BSA (1%) and PMSF (1 mM), incubated at 4° C overnight and then stored at −20° C. prior to ELISA. For each sample the endpoint titer was calculated as the dilution producing the same optical density as the undiluted pre-immune fecal extract.

Immunisations

Groups of seven or eight mice (female, C57 BL/6, 8 weeks) were dosed orally, intranasally or by the intraperitoneal route with suspensions of either spores expressing CotB-TTFC (RH103) or control, non-expressing, spores (strain PY79). For both oral and intranasal dosings mice were lightly anesthetised with halothane. Oral and intra-nasal routes employed a multiple dosing regime used previously for optimal mucosal immunisations (6, 5). A naïve, non-immunised control group was included. Oral dosings also included a group of seven mice receiving 4 μg/dose of purified TTFC protein.
a) Oral immunisations contained 1.67×10¹⁰ spores in a volume of 0.15 ml and were administered by intra-gastric gavage on days 0, 2, 4, 18, 20, 22, 34, 35 and 36. Serum samples were collected on days—1, 17, 33 and 54 and faeces on days—2, 17, 33 and 52.
b) Intranasal immunisations used doses of 1.11×10⁹ spores in a volume of 20 μl and were administered using a micropipette on days 0, 2, 16, 17, 30 and 31. Serum samples were collected on days—1, 15, 29 and 48. Faeces was collected on days—1, 15, 29 and 47.
c) Immunisations via the intra-peritoneal route contained 1.5×10⁹ spores in a volume of 0.15 ml administered on days 0, 14 and 28. Serum samples were taken on days—1, 7, 22, 36 and 43.
   Tetanus Toxin Challenge

On day 60 after the primary, oral immunisation, RH103-immunised mice were injected subcutaneously with a challenge dose of tetanus toxin equivalent to 10 or 20 LD₅₀. The purified toxin (20 μg protein/Lf; Lf=flocculation unit) was suspended in sterile 0.9% NaCl. The LD₅₀ of tetanus toxin was first determined experimentally to be 0.0003 Lf (i.e., 1 LD₅₀=6 ng of protein) and the injection volume was 200 μl/mouse. Animals were closely monitored for signs of tetanus, and mice that developed symptoms of paralysis were humanely euthanised. Individuals showing no symptoms after 14 days were considered immune. Mice that received oral immunisation of TTFC purified protein were challenged with 10 LD₅₀. Naïve mice or mice immunised with PY79 spores were challenged with 2 LD₅₀.

Extraction of Spore Coat Proteins

Spore coat proteins were extracted from suspensions of spores at high density (>1×10¹⁰ spores/ml) using an SDS-DTT extraction buffer as described in detail elsewhere (1).

Extracted proteins were assessed for integrity by SDS-PAGE and for concentration using the BioRad DC Protein Assay kit.

Dissemination Experiments

Balb/c mice (female, 5 weeks) were dosed orally with 1×10⁹ spores/dose of strain SC2362 (rrnO-lacZ cat) consecutively for five days. SC2362 provided a Lac phenotype recognisable as blue colonies on nutrient agar (containg Xgal) as well as chloramphenicol resistance (5 μg/ml; encoded by the cat gene). At time points thereafter groups of four mice were sacrificed and sample organs and tissues dissected in the following sequence.

First, fresh fecal pellets were collected after which the animal was killed by inhalation of CO₂ and decontaminated with 70% alcohol. Peritoneal macrophages were collected by injecting 3 ml sterile PBS into the abdominal cavity, followed by gentle massaging. The peritoneal exudate was then collected using a 21 gauge needle and syringe and processed immediately. The abdominal cavity was then opened and liver excised. The intestine was unbundled and the mesentery removed. Next the spleen and kidneys were collected after which the Peyer's patches located and excised avoiding contamination from the intestinal lumen contents (surrounding tissues were also excised as negative controls). Finally, cervical and submandibular glands were collected. Sterile dissecting instruments were changed between organs. Samples were homogenised by vortexing in 1 ml PBS with 3 ml of glass beads (a mixture of 2 mm and 4 mm diameter), then plated for CFU immediately (on nutrient agar containing Xgal and chloramphenicol) to establish total viable counts or heat-treated (65° C., 1 h) prior to plating to determine spore counts.

### RESULTS

Surface Expression of a Heterologous Antigen on the Spore Surface

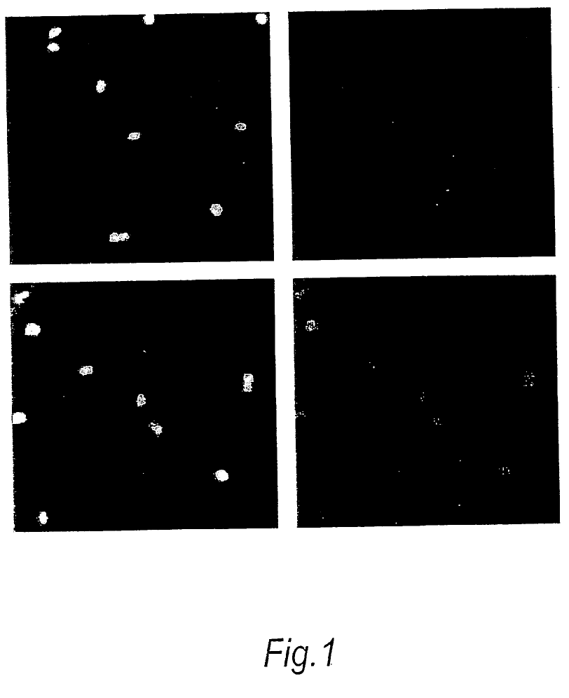

Recombinant spores (RH103) expressing TTFC fused as a chimera to the spore coat protein CotB have been described elsewhere (3). Before assessing the immune responses to spores expressing TTFC we verified that TTFC was surface exposed by immunofluorescence as shown in FIG. 1. Using polyclonal sera against TTFC and CotB we could detect TTFC in sporulating cultures harvested at hour 5 following the initiation of spore formation. We could also detect CotB and TTFC at hours 4 and 6 (data not shown). Sporangial cells were used for labelling since other studies have shown that high levels of background labelling prohibit the use of released endospores (4). Our results showed intact ovoid forespores that labelled with anti-TTFC serum. Labelling with CotB antiserum detected CotB in both recombinant and non-recombinant spores (Panels A and C).

Serum Anti-TTFC Responses Following Intra-peritoneal Injection of Recombinant Spores Expressing TTFC

Before commencing oral and intranasal immunisations we used a pilot study to evaluate the immunogencity of recombinant spores. Groups of eight C57 mice were injected (intra-peritoneal) with recombinant or non-recombinant spores. Our immunisation schedule used a standard regime of three injections (containing 1.5×10⁹ spores/dose) of either recombinant RH103 spores (expressing hybrid CotB-TTFC) or non-recombinant PY79 spores. In a previous study (3) RH103 spores were shown to carry approximately 9.75×10⁻⁵ pg of TTFC polypeptide/spore so our immunising dose would contain 0.15 μg of TTFC. Immunisation with RH103 spores resulted in peak anti-TTFC IgG titres of 1.5×10³ determined by indirect ELISA (data not shown), significantly different (p<0.05) from control groups (1.1×10² for PY79 and 0.8×10¹ for naïve), demonstrating that TTFC was stably expressed and appropriately immunogenic when displayed on the spore surface.

Serum Anti-TTFC Responses Following Oral and Intranasal Immunisation

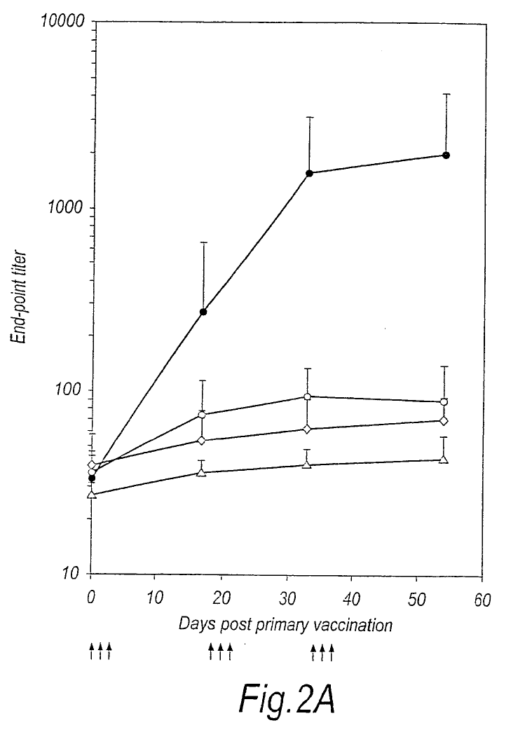

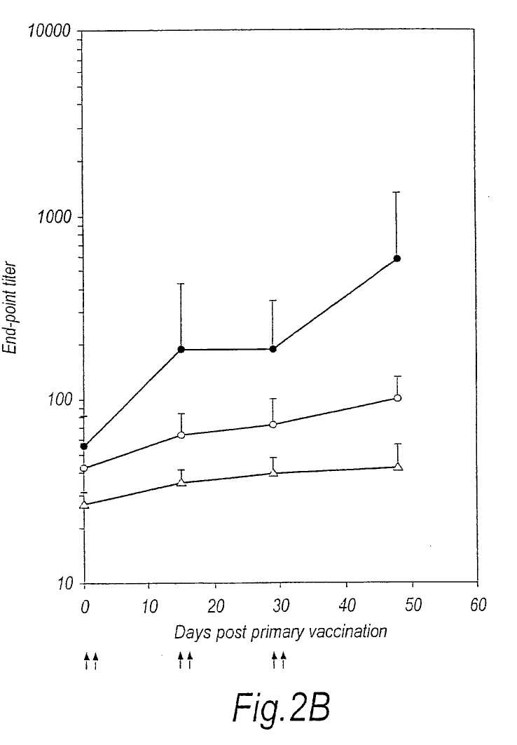

To test for induction of mucosal and systemic responses, groups of seven mice were immunised either orally (1.67×10¹⁰ spores/dose; 1.65 μg TTFC/dose) or intranasally (1.11×10⁹ spores/dose; 0.11 μg TTFC/dose). Note that technically, larger doses could not be given by the nasal route. As shown in FIG. 2A oral immunisation of mice with RH103 (CotB-TTFC) spores gave titres greater than 1×10³ by day 33, significantly above (p<0.05) those of mice dosed with non-recombinant spores (PY79), mice given purified TTFC protein (4 μg/dose), or the control naïve group. TTFC protein was not used as a control for the intra-nasal route since previous work has shown that TTFC delivered nasally (with a low dose, i.e. less than 10 μg/dose) is not immunogenic (8).

Somewhat lower levels of TTFC-specific IgG end point titers were found at day 48 following intranasal immunisation (FIG. 2B). Our data showed that by either route, the titers for the naïve and non-recombinant immunisations were not significantly different (p>0.05). Groups administered with spores expressing TTFC fused to CotB responded with significantly higher TTFC-specific IgG titers than their corresponding control groups (p<0.05) from day 33 onwards for oral groups and from day 29 for intranasal groups. In work not shown we have also found that RH103 spores dosed orally with or without cholera toxin (type Inaba 569B, 0.33 μg/dose) gave no significant difference in anti-TTFC IgG titres.

Serum Anti-TTFC Antibody Isotypes

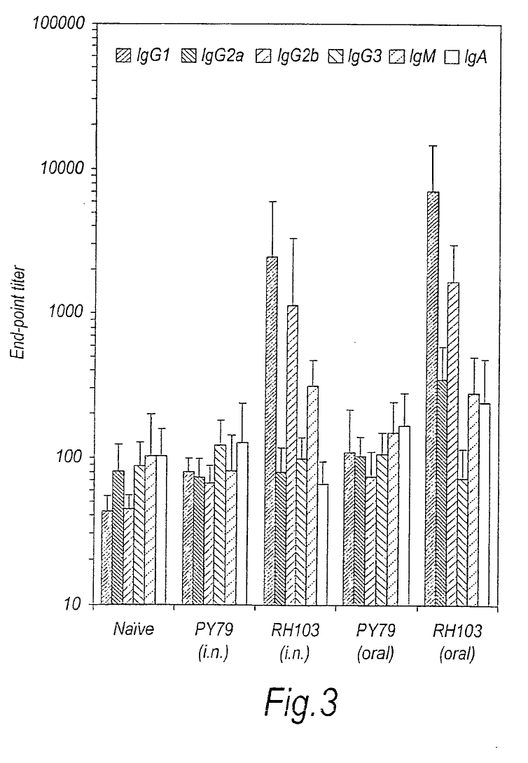

Sera from mice immunised mucosally was also examined for the presence of TTFCspecific IgG, IgA and IgM antibody isotypes as well as the IgG1, IgG2a, IgG2b and IgG3 subclasses (FIG. 3). Mice immunised orally with RH103 spores expressing CotB-TTFC showed high levels, at day 54, of the IgG1 and IgG2b isotypes. For the IgG1, IgG2a and IgG2b subclasses the mean titers were significantly different from baseline titers in the two control groups, i) naïve mice and ii) mice immunised with non-recombinant spores (p<0.05). Little change was observed with the IgG3, IgM and IgA subclasses. In mice immunised intranasally, the sera at day 48 showed a predominance of the IgG1, IgG2b and the IgM subclasses. For these subclasses, titers were significantly higher than in the control groups (p<0.05). In contrast, no significant variation (p>0.05) in any of the isotypes was seen between groups administered with non-recombinant spores and the naïve group.

Mucosal Anti-TT]F¢C IgA Responses

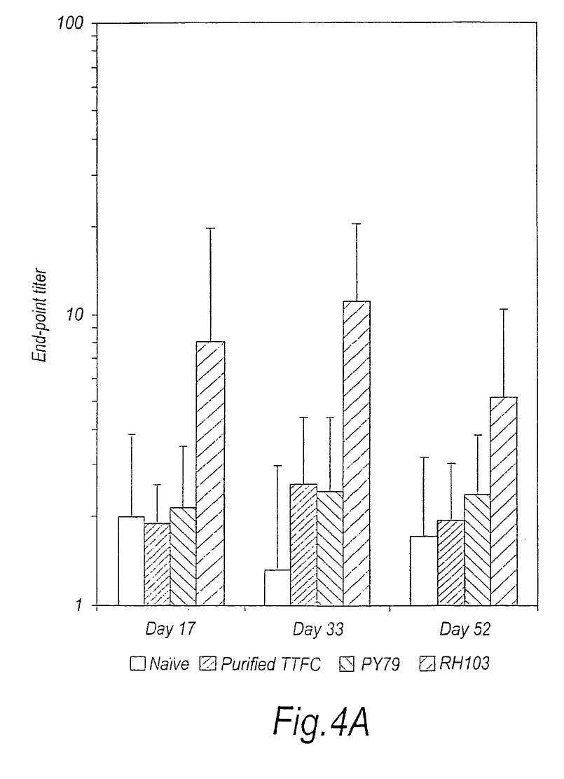

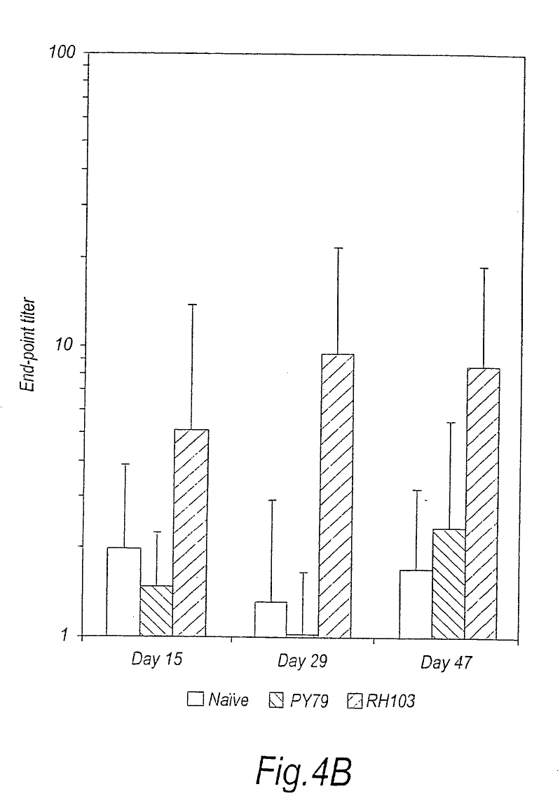

Fresh fecal pellets from mice immunised orally or intranasally was tested for the presence of TTFC-specific secretory IgA (sIgA) by ELISA (FIG. 4). Immunisation with spores expressing CotB-TTFC by either route elicited clear TTFC-specific sIgA responses. In groups of mice immunised orally or intranasally TTFC-specific sIgA titers peaked at day 33 (FIGS. 4A & 4B). The end-point titers of fecal TTFC-specific sIgA were shown to be significantly higher than the control groups (p<0.05) while there was no significant difference between the control groups (non-recombinant spores and naïve groups; p>0.05).

Protection Against Tetanus Toxin Challenge After Oral Immunisation

The high serum IgG titres (>10³) observed following oral immunisation were at potentially protective levels. In order to test the biological activity of the elicited antitoxin response and the associated level of protection, mice orally immunised with CotB-TTFC expressing B. subtilis spores (RH103) were challenged with a lethal dose of tetanus toxin (10 or 20 LD₅₀) given subcutaneously (Table 2).

TABLE 2
Protection of mice against lethal systemic challenge with
tetanus toxin after oral immunization.

| | Toxin challenge | Survival/ |
|---|---|---|
| Group | dose (LD₅₀) | Total |
| Naïve | 2 | 0/5 |
| PY79 spores | 2 | 0/8 |
| TTFC purified protein | 10 | 0/8 |
| RH103 (CotB-TTFC) spores | 10 | 8/8 |
| RH103 (CotB-TTFC) spores | 20 | 7/8 |

Table 2 shows the result of treatment of groups of eight mice which were immunized orally with 1.67×10¹⁰ spores of B. subtilis or 4 μg of TTFC purified protein on days 0, 2, 4, 18, 20, 22, 34, 35 and 36 before being injected subcutaneously with a challenge dose of tetanus toxin on day 60. Individuals developing no symptoms after 14 days were considered immune.

Mice were fully protected against the challenge of 10 LD₅₀. Out of eight mice challenged with 20 LD₅₀, one mouse had clear symptoms after 72 h. All naïve mice and mice immunised with wild type B. subtilis spores (PY79) showed clear tetanus signs within 72 h after the challenge of 2 LD₅₀. Oral immunisation with TTFC purified protein (4 μg/dose) gave no protection against 10 LD₅₀ and all mice showed clear symptoms of tetanus within 24 h. The systemic antibody responses elicited via oral immunisation with B. subtilis spores expressing CotB-TTFC were therefore protective.

Anti-spore Responses

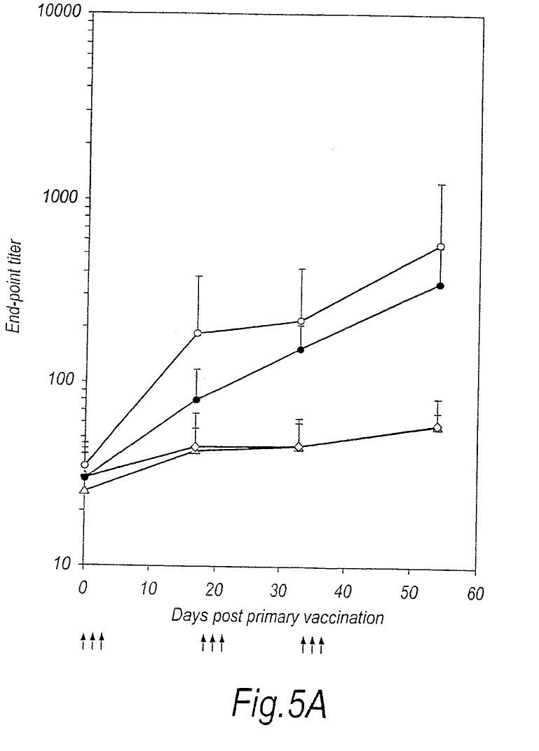

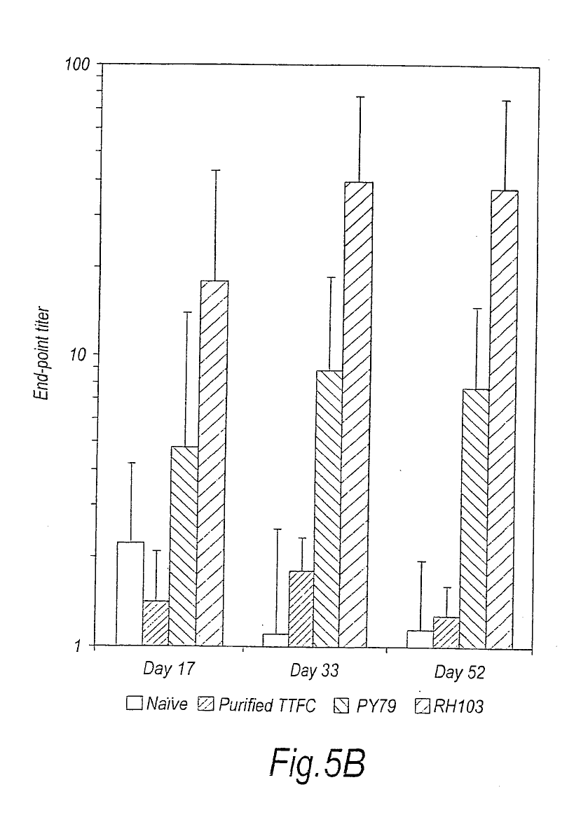

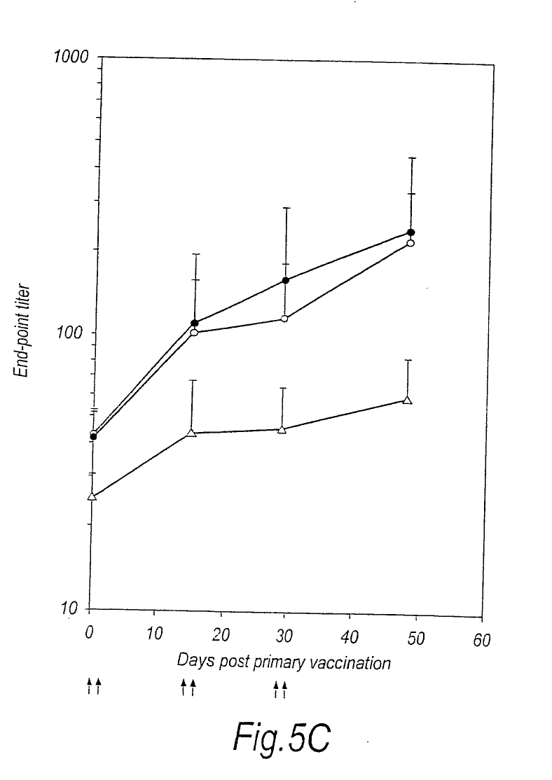

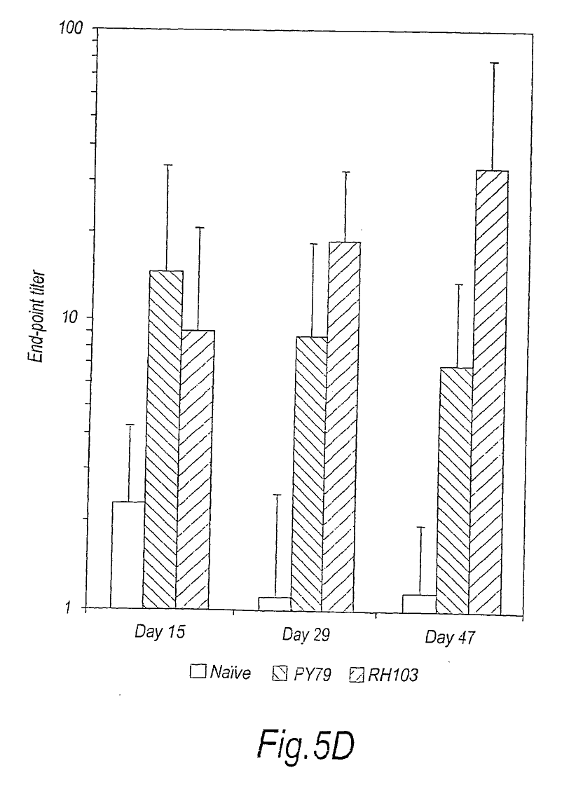

In addition to anti-TTFC responses, anti-spore IgG and sIgA responses following oral and intransal immunisation were determined (FIG. 5). Oral immunisation with both CotB-TTFC expressing spores (RH103) and non-recombinant spores (PY79) produced systemic spore coat-specific IgG levels (FIG. 5A) that were significantly higher than the naïve group (p<0.05). Lower, but still significant levels (p<0.05) of spore coat specific IgG titers were observed following intranasal immunisation whether recombinant or non-recombinant spores were used (FIG. 5C).

Spore coat-specific sIgA levels observed in the faeces of orally immunised mice (FIG. 5B) showed substantial responses against spores. These levels were significantly higher (p<0.05) than when non-recombinant spores were used for immunisation. When the intranasal route (FIG. 5D) was used for immunisation a similar profile of spore coat-specific sIgA levels was observed with a reduction of IgA levels over time in mice dosed with non-recombinant spores. Again, the levels of spore coat-specific sIgA were significantly higher than in naïve mice (p<0.05).

Dissemination of Spores

Inbred Balb/c mice were dosed daily with 1×10⁹ spores/dose for five consecutive days. Pilot studies had shown that this consecutive dosing regime was sufficient to establish recoverable and statistically relevant counts. At time points following the final dosing groups of four mice were sacrificed and key lymphoid organs dissected. In addition faeces was collected, homogenised and counts determined. Total viable counts and heat resistant counts were determined in homogenised tissues and faeces. Recovered viable counts are given in Table 3 and show recovery of bacteria from intestinal Peyer's patches and mesenteric lymph nodes suggesting interaction with the GALT.

TABLE 3

| | | Day | | | | | |
|---|---|---|---|---|---|---|---|
| Organ | | 1 | 2 | 3 | 5 | 7 | 9 |
| Faeces | Tot. | 1.68 × 10⁶ ± | 5.25 × 10⁵ ± | 1.79 × 10⁵ ± | 4.61 × 10⁴ ± | 2.99 × 10⁴ ± | 1.44 × 10³ ± |
| per g | | 1.1 × 10⁶ | 4.5 × 10⁵ | 1.1 × 10⁵ | 0.7 × 10⁴ | 1.4 × 10⁴ | 1.1 × 10³ |
| | Spores | 1.73 × 10⁶ ± | 7.96 × 10⁵ ± | 1.36 × 10⁵ ± | 4.41 × 10⁴ ± | 1.21 × 10⁴ ± | 1.08 × 10³ ± |
| | | 1.0 × 10⁶ | 6.7 × 10⁵ | 1.0 × 10⁵ | 0.8 × 10⁴ | 1.1 × 10⁴ | 1.0 × 10³ |
| PP/MLN | Tot. | 227 ± 134 | 27 ± 18 | NS | NS | NS | NS |
| | Spores | 166 ± 124 | 27 ± 18 | NS | NS | NS | NS |
| SMG/CLN | Tot. | 105 ± 71 | 117 ± 9 | 15 ± 10 | 405 ± 59 | 126 ± 39 | 29 ± 20 |
| | Spores | 42 ± 29 | 65 ± 26 | 22 ± 18 | 110 ± 87 | 39 ± 15 | 19 ± 15 |
| Spleen | Tot. | NS | NS | 25 ± 19 | 0 | NS | NS |
| | Spores | 0 | NS | NS | NS | NS | NS |
| PM | Tot. | 75 ± 40 | 45 ± 17 | 30 ± 27 | 56 ± 50 | 30 ± 27 | NS |
| | Spores | 45 ± 31 | 45 ± 38 | 36 ± 24 | 33 ± 30 | NS | NS |
| Liver | Tot. | NS | ND | ND | NS | NS | 0 |
| | Spores | NS | ND | ND | ND | ND | 0 |
| Kidneys | Tot. | NS | ND | ND | NS | NS | NS |
| | Spores | NS | ND | ND | NS | NS | NS |

Table 3 shows the results of the treatment of groups of four Balb/c mice dosed orally with 1×10⁹ spores of B. subtilis strain SC2362 (rrnO-lacZ) for five consecutive days (total dose, 5×10⁹). Results are given as mean numbers of colony forming units per mouse organ taken at the indicated times after the last day of dosing. Expressed as total counts (no heat treatment) and spore counts (samples treated 65° C., 1 h). ND, not determined; NS, not significant (<10 viable units per sample). Data are presented as arithmetic means±standard deviation.

In Table 3, PP/MLN is an abbreviation for Peyer's patches and mesenteric lymph nodes; SMG/CLN is an abbreviation for submandibular gland and cervical lymph nodes and PM is an abbreviation for peritoneal macrophages.

Most interesting was the recovery of viable counts in the submandibular glands and cervical lymph nodes with no recovery of significant counts from the liver and spleen. Recovery of bacteria from head and neck tissues with little or no recovery from widely disseminated systemic sites suggests that spores may have crossed the rhinopharanygeal mucosa. Counts in faeces declined steadily as bacteria were cleared from the GIT although little difference between total and spore counts was observed.

### REFERENCES

(1) Nicholson, W. L., and P. Setlow. 1990. Sporulation, germination and outgrowth., p. 391-450. In C. R. Harwood., and S. M. Cutting. (eds), Molecular Biological Methods for Bacillus. John Wiley & Sons Ltd., Chichester, UK
(2) Youngman, P., J. Perkins, and R. Losick. 1984. Construction of a cloning site near one end of Tn917 into which foreign DNA may be inserted without affecting transposition in Bacillus subtilis or expression of the transposon-borne erm gene. Plasmid 12:1-9.
(3) Isticato, R., G. Cangiano, H. T. Tran, A. Ciabattini, D. Medaglini, M. R. Oggioni, M. De Felice, G. Pozzi, and E. Ricca. 2001. Surface display of recombinant proteins on Bacillus subtilis spores. J. Bacteriol. 183:6294-6301.
(4) Harry, E. J., K. Pogliano, and R. Losick. 1995. Use of immunoflurescence to visualize cell-specific gene expression during sporulation in Bacillus subtilis. J. Bacteriol. 177:3386-3393.
(5) Robinson, K., L. M. Chamberlain, K. M. Schofield, J. M. Wells, and R. W. F. Le Page. 1997. Oral vaccination of mice against tetanus with recombinant Lactococcus lactis. Nat. Biotechnol. 15:653-657.
(6) Challacombe, S. J. 1983. Salivary antibodies and systemic tolerance in mice after oral immunisation with bacterial antigens. Ann. N.Y. Acad. Sci. 409:177-192.
(7) Driks, A. 1999. Bacillus subtilis spore coat. Microbiol. Mol. Biol. Rev. 63:1-20.
(8) Douce, G., C. Turcotte, I. Cropley, M. Roberts, M. Pizza, M. Domenghini, R. Rappuoli, and G. Dougan. 1995. Mutants of Escherichia coli heat-labile toxin lacking ADP-ribosyltransferase activity act as nontoxic, mucosal adjuvants. Proc. Natl. Acad. Sci. U. S. A. 92:1644-1648.
(9) Hoa, T. T., L. H. Duc, R. Isticato, L. Baccigalupi, E. Ricca, P. H. Van, and S. M. Cutting. 2001. The fate and dissemination of B. subtilis spores in a murine model. Appl. Environ. Microbiol. 67:3819-3823. inserted without affecting transposition in Bacillus subtilis or expression of the transposon-borne erm gene. Plasmid 12:1-9.
(3) Isticato, R., G. Cangiano, H. T. Tran, A. Ciabattini, D. Medaglini, M. R. Oggioni, M. De Felice, G. Pozzi, and E. Ricca. 2001. Surface display of recombinant proteins on Bacillus subtilis spores. J. Bacteriol. 183:6294-6301.
(4) Harry, E. J., K. Pogliano, and R. Losick. 1995. Use of immunoflurescence to visualize cell-specific gene expression during sporulation in Bacillus subtilis. J. Bacteriol. 177:3386-3393.
(5) Robinson, K., L. M. Chamberlain, K. M. Schofield, J. M. Wells, and R. W. F. Le Page. 1997. Oral vaccination of mice against tetanus with recombinant Lactococcus lactis. Nat. Biotechnol. 15:653-657.
(6) Challacombe, S. J. 1983. Salivary antibodies and systemic tolerance in mice after oral immunisation with bacterial antigens. Ann. N.Y. Acad. Sci. 409:177-192.
(7) Driks, A. 1999. Bacillus subtilis spore coat. Microbiol. Mol. Biol. Rev. 63:1-20.
(8) Douce, G., C. Turcotte, I. Cropley, M. Roberts, M. Pizza, M. Domenghini, R. Rappuoli, and G. Dougan. 1995. Mutants of Escherichia coli heat-labile toxin lacking ADP-ribosyltransferase activity act as nontoxic, mucosal adjuvants. Proc. Natl. Acad. Sci. U. S. A. 92:1644-1648.
(9) Hoa, T. T., L. H. Duc, R. Isticato, L. Baccigalupi, E. Ricca, P. H. Van, and S. M. Cutting. 2001. The fate and dissemination of B. subtilis spores in a murine model. Appl. Environ. Microbiol. 67:3819-3823.

1-17. (canceled)

18. A spore genetically modified with genetic code comprising at least one genetic construct encoding an antigen and a spore coat protein as a chimeric gene, said genetically modified spore having said antigen expressed as a fusion protein with said spore coat protein for use in oral or intranasal or rectal administration for therapeutic treatment.

19. A spore as claimed in claim 18, wherein the spore is of Bacillus species.

20. A spore as claimed in claim 18, wherein the genetic construct comprises at least part of a spore coat protein gene and at least part of an antigen gene, in the form of a chimeric gene.

21. A spore as claimed in claim 18, wherein the antigen gene is located at the 3′ end of the spore coat protein gene.

22. A spore as claimed in claim 18, wherein the genetic construct comprises a spore coat promoter at the 5′ end of the chimeric gene.

23. A spore as claimed in claim 22, wherein the antigen is at least one of tetanus toxin fragment C or labile toxin B subunit.

24. A spore as claimed in claim 18, wherein the spore coat protein is selected from the group consisting of cotA, cotB, cotC, cotD, cotE, cotF, cotG, cotH, cotJA, cotJC, cotM, cotSA, cotS, cotT, cotV, cotW, cotX, cotY and cotZ.

25. A spore as claimed in claim 24, wherein the spore is heat inactivated so that in use it does not germinate into a vegetative cell.

26. A spore as defined in claim 18 for use in the treatment of a medical condition.

27. A composition comprising at least two different spores as defined in claim 18, wherein said at least two different spores express at least two different antigens.

28. A composition as defined in claim 27, wherein the composition further comprises a pharmaceutically acceptable excipient or carrier.

29. A composition comprising a spore as defined in claim 18 in association with a pharmaceutically acceptable excipient or carrier for use in oral or intranasal or rectal administration for therapeutic treatment.

30. A composition comprising a spore as defined in claim 26 in association with a pharmaceutically acceptable excipient or carrier for use in oral or intranasal or rectal administration for therapeutic treatment.

31. A composition as defined in claims 27, 28 or 29, for use in treatment of a medical condition, preferably the medical condition is inflammation, pain, a hormonal imbalance and/or an intestinal disorder.

32. Use of a spore as defined in claim 18 in the manufacture of a medicament for use in the treatment of a medical condition, preferably the medical condition is inflammation, pain, a hormonal imbalance and/or an intestinal disorder.

33. Use of a spore as defined in claim 26 in the manufacture of a medicament for use in the treatment of a medical condition, preferably the medical condition is inflammation, pain, a hormonal imbalance and/or an intestinal disorder.

34. A method of medical treatment, which method comprises the steps of
a) administering a spore as defined in claim 18 to a human or animal in need of medical treatment by an oral, intra-nasal or rectal route;
b) said genetically modified spore eliciting an immune response for use in the prevention of a disease.

35. A method of medical treatment, which method comprises the steps of
a) administering a spore as defined in claim 26 to a human or animal in need of medical treatment by an oral, intra-nasal or rectal route;
b) said genetically modified spore eliciting an immune response for use in the prevention of a disease.

36. A method of producing a genetically modified spore, which method comprises the steps;
producing genetic code comprising at least one genetic construct encoding an antigen and a spore coat protein as a chimeric gene;
using said at least one genetic construct to transform a vegetative mother cell;
inducing said transformed mother cell to sporulate; and
isolating the resulting genetically modified spores.
